(19) United States
(12) Patent Application Publication
(10) Pub. No.: US 2016/0213593 A1
(43) Pub. Date: Jul. 28, 2016

(54) Flexible Nail Glue Composition and Laminate

(71) Applicant: Chemence, Inc., Alpharetta, GA (US)

(72) Inventors: Peter D. Battisti, Alpharetta, GA (US); Krasimir G. Chorbadzhiev, Alpharetta, GA (US)

(73) Assignee: Chemence, Inc., Alpharetta, GA (US)

(21) Appl. No.: 15/006,353

(22) Filed: Jan. 26, 2016

Related U.S. Application Data
(60) Provisional application No. 62/107,641, filed on Jan. 26, 2015.

(51) Int. Cl. A61K 8/81; A61Q 3/00; A61K 8/37; A45D 31/00

(52) U.S. Cl. CPC A61K 8/8152; A45D 31/00; A61Q 3/00; A61K 8/37

(57) ABSTRACT

A laminate of an artificial nail bound to a natural nail with a layer of a cyanoacrylate adhesive formulation containing a thickener, a stabilizer, and a plasticizer is as strong and durable as similar laminates but is flexible and pliable enough to bend without cracking.

### CROSS-REFERENCES TO RELATED APPLICATIONS

This is a non-provisional application for patent claiming priority of U.S. provisional application No. 62/107,641.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### REFERENCE TO A BIOLOGICAL SEQUENCE LISTING

Not applicable.

### BACKGROUND OF INVENTION

1. Field of the Invention

This invention is in the field of adhesives, also in the field of cosmetics.

2. Description of the Related Art:

An important use of cyanoacrylate adhesive, i.e., “superglue,” is to bond artificial nails onto natural nails to improve their appearance and durability. (Herein, “nail” refers to fingernails and toenails.) The chief complaint with nail glues is called “pop off,” where the false nail comes off the real nail.

Part of this problem is that while both the natural nail and the artificial nail are flexible, the glue used to bond these two flexible substrates together is brittle. Thus as the nails flex, the glue cracks and this weakens the bond. The cracks in the glue create fissures where moisture and oils can wick in and hasten de-lamination of the artificial nail from the natural nail. The intrusion of moisture also allows microbial growth.

In reference to improving the flexibility of cyanoacrylate adhesives, the following additives in have been used to render them more flexible: triacetin (glyceryl triacetate) (used in FN adhesives); tributyl citrate and acetyl tributyl citrate (used in medical adhesives); rubber, e.g., Vamac G; mixed monomer formulations, e.g., octyl, methoxy, or propoxyethyl cyanoacrylate; and incorporation of non-cyanoacrylate esters, e.g., octylphthalate, dibutyl phthalate, butyl cyanoacetate, etc. U.S. Pat. No. 6,977,278 is a good reference as to additives for improving the flexibility of cyanoacrylate adhesives.

Flexibility, however, is not the only parameter to consider in solving the “pop off” problem. The strength and durability of the adhesive bond must be maintained, and additives may affect these adversely. The modified glue must be compatible with both the natural nail material and the artificial nail material Finally, the additive must be approved for human use.

### BRIEF DESCRIPTION OF THE INVENTION

### Objects of the Invention

There has been a long-felt and unmet need for nail glues that resist “pop off” without otherwise interfering with the bond between the natural and artificial nails or interfering with other desirable properties.

### SUMMARY OF THE INVENTION

It has been discovered that the addition of plasticizer, more specifically triacetin, CAS No. 102-30 76-1, to a cyanoacrylate adhesive formulation also containing a thickener and a stabilizer, creates a nail glue which is as strong and durable as existing glues but, in a thin layer such as applied as an adhesive, is flexible and pliable enough to bend with the natural and artificial nails without cracking.

### DETAILED DESCRIPTION OF THE INVENTION

The general form of the instant invention is a mixture of a cyanoacrylate monomer adhesive with a thickener, a plasticizer, and a stabilizer applied in a thin layer between an artificial nail and a natural nail. Preferably, the adhesive should be a low molecular weight aliphatic cyanoacrylate combined with sufficient plasticizer, namely, triacetin, together with a small amount of stabilizer. More preferably, the formula should comprise triacetin in the range of 5 to 20% by weight combined with thickener in the range of 2 to 8%, less than 1% BHA, the balance cyanoacrylate monomer. Still more preferably, the thickener should be polymethyl methacrylate (PMMA), and the monomer should be ethyl cyanoacrylate. Still more preferably, the formula should comprise triacetin in the range of 10 to 15%, PMMA in the range of 4 to 6%, BHA in the range of 0.1 to 0.2%, the balance ethyl cyanoacrylate. The formula of the most preferred embodiment is as listed below:

| Formulation 1 | CAS # | Percent, w/w |
|---|---|---|
| Ethyl cyanoacrylate | 7085-85-0 | 82.74 |
| Polymethyl methacrylate (PMMA) | 9011-14-7 | 4.59 |
| Triacetin (glyceryl triacetate)* | 102-76-1 | 12.50 |
| Butylated hydroxyanisole (BHA) | 25013-16-5 | 0.17 |
| TOTAL | | 100.00 |

*Triacetin is generally recognized as safe (GRAS) by the U.S. Food and Drug Administration for use in food and cosmetics, and is lawful for use in certain food contact applications in the European Union (EU No. 10/2011).

Testing by the inventor has shown that the formulation shown in the above table is unexpectedly superior to nail glues containing other plasticizers in preventing “pop off.”

1. A laminate, comprising:
a natural nail;
an artificial nail; and
a layer of a composition, interposed therebetween, comprising:
at least one cyanoacrylate monomer;
at least one thickener; and
at least one plasticizer.

2. The laminate of claim 1, in which:
said at least one plasticizer is triacetin.

3. The laminate of claim 2, in which:
triacetin is present in the range of 10 to 15% w/w of said composition.

4. The laminate of claim 2, in which:
triacetin is present in the range of 3 to 6% w/w of said composition.

5. The laminate of claim 2, in which:
triacetin is present in the range of 4 to 5% w/w of said composition.

6. The laminate of claim 2, in which:
triacetin is present at a concentration of about 4.5% w/w of said composition.

7. The laminate of claim 2, in which:
said at least one cyanoacrylate monomer is ethyl cyanoacrylate; and
said at least one thickener is PMMA.

8. The laminate of claim 7, in which:
triacetin is present in the range of 10 to 15% w/w of said composition.

9. The laminate of claim 7, in which:
triacetin is present in the range of 3 to 6% w/w of said composition.

10. The laminate of claim 7, in which:
triacetin is present in the range of 4 to 5% w/w of said composition.

11. The laminate of claim 7, in which:
triacetin is present at a concentration of about 4.5% w/w of said composition.

12. The laminate of claim 1, in which said composition comprises:
up to about 1% stabilizer;
said thickener is 4 to 6% w/w PMMA;
said plasticizer is 10 to 15% w/w triacetin; and
the balance weight percentage is ethyl cyanoacrylate monomer.

13. The laminate of claim 1, in which said composition comprises:
up to about 1% stabilizer;
said thickener is 4 to 6% w/w PMMA;
said plasticizer is 3 to 6% w/w triacetin; and
the balance weight percentage is ethyl cyanoacrylate monomer.

14. The laminate of claim 1, in which said composition comprises:
up to about 1% stabilizer;
said thickener is 4 to 6% w/w PMMA;
said plasticizer is 4 to 5% w/w triacetin; and
the balance weight percentage is ethyl cyanoacrylate monomer.

15. The laminate of claim 1, in which said composition comprises:
up to about 1% stabilizer;
said thickener is 4 to 6% w/w PMMA;
said plasticizer is about 4.5% w/w triacetin; and
the balance weight percentage is ethyl cyanoacrylate monomer.

16. The laminate of claim 1, in which said composition comprises:
about 83% cyanoacrylate monomer;
about 4.5% PMMA; and
about 12.5% triacetin.
